(19) United States
(12) Patent Application Publication
(10) Pub. No.: US 2015/0283194 A1
(43) Pub. Date: Oct. 8, 2015

(54) METHOD FOR TREATING PAIN WITH ASTER EXTRACT

(71) Applicant: MEDICAL AND PHARMACEUTICAL INDUSTRY TECHNOLOGY AND DEVELOPMENT CENTER, Wugu, New Taipei (TW)

(72) Inventors: Jui-Ching Chen, New Taipei City (TW); Che-Yi Lin, Tainan City (TW); Yu-Hsiang Huang, Chiayi City (TW); Ming-Yu Chao, New Taipei City (TW); Chih-Sheng Hung, New Taipei City (TW)

(21) Appl. No.: 14/436,332

(22) PCT Filed: Dec. 18, 2013
(86) PCT No.: PCT/CN2013/089884

§ 371 Date: Apr. 16, 2015

Related U.S. Application Data
(60) Provisional application No. 61/738,395, filed on Dec. 18, 2012.

(51) Int. Cl. A61K 36/28; A61K 31/485

(52) U.S. Cl. CPC A61K 36/28; A61K 31/485

(57) ABSTRACT

Disclosed herein is the use of an extract from an Aster species for the preparation of a medicament for the treatment of pain. The extract is extracted from fresh and/or dried roots and rhizomes of a Tatarian aster (Aster tartaricus) plant, in which the extraction is performed by use of water or 10-95% (v/v) ethanol as an extractant to obtain an extraction mixture. In some embodiments, the extraction mixture is further subject to column chromatography, in which the cloumn was eluted in sequence with water, and at least one eluent other than water.

### BACKGROUND OF THE INVENTION

1. Field of the Invention

The present disclosure relates to the treatment of pain. More particularly, the disclosed invention relates to the use of an extract obtaining from Aster spp. as an analgesic in the treatment of pain.

2. Description of Related Art

Control of pain, especially chronic pain, is a significant health issue. According to a report from the American Academy of Pain Medicine, the annual cost for health care and lost productivity related to pain is at least $560-$635 billion in the U.S.

Analgesics are agents that relieve pain by acting centrally or peripherally to elevate pain threshold, preferably without disturbing consciousness or altering other sensory functions, and has accordingly developed a number of opiate and opioid analgesics having diverse pharmacological actions. While opioid analgesics remain the mainstay for pain treatment, prolonged use of these drugs leads to tolerance which results in frequent dose escalation and increased side effects, such as opioid-induced constipation (OK), altered cognitive state and inadequate pain control, and possibly drug dependence.

There exist in this art a need of an improved pain therapy that may enhance the efficacy of the existing opioid-based treatment, or may act as an alternative to the current opioid-basal treatment.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is based, at least in part, on the discovery that the solvent extract form an Aster tartaricus (Tatarian aster) plant may act as an effective analgesia in a subject. Therefore, one aspect of the present invention pertains to a method for treating pain in a subject by administering to the subject an effective amount of a solvent extract of Tatarian aster (hereinafter, the solvent aster extract). In another aspect, the present invention pertains to the use of the solvent aster extract for the preparation of a medicament for use in the treatment of pain.

According to embodiments of the present invention, the aster extract is prepared from a component of the plant of Tatarian aster. The plant component for use in the present invention may be a fresh or dried material collected from the rhizome and/or root of a Tatarian aster plant.

According to embodiments of the present invention, the preparation of the aster extract comprises an extraction step, in which the component collected from the Tatarian aster is extracted with an extractant to produce an extraction mixture. Extractants suitable for use herein may be water or 10-95% (v/v) ethanol.

In one embodiment, the amount of the solvent of the extraction mixture is altered so that a precipitate suitable for use in the preparation of the present medicament is formed. For example, when the alcoholic solution is used as the extractant, water is added into the extraction mixture to give a precipitate. Alternatively, when water is used as the extractant, alcoholic solution is added into the extraction mixture to give a precipitate. Optionally, the precipitate may be dried to yield extract powders.

Alternatively, in some embodiments, the extraction mixture is subjected to a column chromatography in which the column is eluted with water followed by at least one eluents to obtain at least one eluate which is suitable for use in the preparation of the present medicament. Non-limiting examples of the eluent include, but are not limited to, 40-95% (v/v) ethanol, acetone, and 40-95% (v/v) ethanol containing 0.1-1% (v/v) formic acid.

Optionally, extraction mixture is filtered, concentrated, and/or dried prior to being subjected to the column chromatography. Still optionally, the eluate is concentrated to reduce the volume of the eluate and remove the eluent therefrom.

According to a first particular embodiment of the present invention, the method of producing the aster extract comprises steps as follows. A dried root or rhizome of a Tatarian aster plant, or a combination of both is extracted with water to produce an extraction mixture. Then the extraction mixture is subjected to a column chromatography by eluting the column in sequence with water and 95% (v/v) ethanol, and an eluate obtained from the column eluted with 95% (v/v) ethanol is collected.

According to a second particular embodiment of the present invention, the method of producing the aster extract differs from the previous embodiment in that the column is first eluted with water, then followed by sequentially with (1) 40% (v/v) ethanol and (2) 95% (v/v) ethanol to obtain an eluate. Other than the elution step, the respective processes of the first and second embodiments are substantially the same. Then, the eluate obtained from the column eluted with 95% (v/v) ethanol is collected.

Another aspect of the present invention pertains to a method for enhancing the analgesic effect of an opioid drug in a subject in need thereof. In practice, the method comprises administering to the subject an analgesically effective amount of the opioid drug and an effective amount of an extract of Tatarian aster (hereafter the aster extract) for enhancing the analgesic effect of the opioid drug in the subject. In still another aspect, the present invention pertains to the use of the solvent aster extract for the preparation of a medicament for use as an adjuvant that enhances the analgesic effect of an opioid drug.

Specifically, the aster extract for use in the present method is prepared in accordance with the preparation process provided in various embodiments of the present invention. According to certain embodiments of the present disclosure, the aster extract is administered prior to the administration of opioid drug.

According to one embodiment of the present invention, the aster extract capable of enhancing the analgesic effect of the opioid drug is prepared by a preparation method comprising the steps as follows. A dried root or rhizome of a Tatarian aster plant, or a combination of both is extracted with 50% (v/v) ethanol to obtain an extraction mixture. Then the extraction mixture is subjected to a column chromatography by eluting the column with water followed by, sequentially, (1) 95% (v/v) ethanol, (2) acetone, (3) 95% (v/v) ethanol containing 0.1% (v/v) formic acid, and (4) 50% (v/v) ethanol containing 0.1% (v/v) formic acid; and the eluates respectively obtained from the column eluted with acetone, 95% (v/v) ethanol containing 0.1% (v/v) formic acid, and 50% (v/v) ethanol containing 0.1% (v/v) formic acid, are collected and combined.

Many of the attendant features and advantages of the present disclosure will become better understood with reference to the following detailed description.

### BRIEF DESCRIPTION OF THE DRAWINGS

The present description will be better understood from the following detailed description read in light of the accompanying drawings, where:

FIG. 1A and FIG. 1B are respectively line graph and bar graph illustrating the experimental result according to one working example of the present disclosure;

FIG. 2A and FIG. 2B are respectively line graph and bar graph illustrating the experimental result according to another working example of the present disclosure;

FIG. 3A and FIG. 3B are respectively line graph and bar graph illustrating the experimental result according to yet another working example of the present disclosure; and

FIG. 4 is a bar graph illustrating the experimental result according to still another working example of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “pain” as used herein generally refers to an unpleasant sensory and emotional experience associated with actual or potential tissue damage caused by or resulting in stimulation of nociceptors in the peripheral nervous system, or by damage to or malfunction of the peripheral or central nervous systems and neural voltage channel transmission.

As used herein, the term “treating” pain encompasses partially or completely preventing, ameliorating, mitigating and/or managing pain. The term “treating” includes,” as used herein refers to application or administration of the aster extract of the present disclosure to a subject, who has a medical condition associated with pain, a symptom of the condition, a disease or disorder secondary to the condition, or a predisposition toward the condition, with the purpose to partially or completely alleviate, ameliorate, relieve, delay onset of inhibit progression of reduce severity of and/or reduce incidence of one or more symptoms or features of a particular disease, disorder, and/or condition associated with pain. Treatment may be administered to a subject who does not exhibit signs of such disease, disorder, and/or condition and/or to a subject who exhibits only early signs of such disease, disorder, and/or condition for the purpose of decreasing the risk of developing pathology associated with the disease, disorder, and/or condition. Treatment is generally “effective” if one or more symptoms or clinical markers are reduced as that term is defined herein. Alternatively, a treatment is “effective” if the progression of a disease is reduced or halted.

The term “effective amount” as used herein refers to the quantity of a component or medicament which is sufficient to yield a desired “effective treatment” as defined hereinabove. The specific therapeutically effective amount will vary with factors such as the particular condition being treated, the physical condition of the patient (e.g., the patient's body mass, age, or gender), the type of mammal or animal being treated, the duration of the treatment, the nature of concurrent therapy (if any), and the specific formulations employed. An effective amount is also one in which any toxic or detrimental effects of the compound or composition are outweighed by the therapeutically beneficial effects. Effective amount may be expressed, for example, as the total mass of the medicament (e.g., in grams, milligrams or micrograms) or a ratio of mass of the medicament to body mass, e.g., as milligrams per kilogram (mg/kg). Persons having ordinary skills could calculate the human equivalent dose (HED) for the medicament (such as the present aster extract) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to an animal including the human species that is treatable with the aster extracts and/or methods of the present disclosure. The term “subject” is intended to refer to both the male and female gender unless one gender is specifically indicated.

The term “analgesia” or “analgesic effect” is used herein to describe states of reduced pain perception, including absence from pain sensations, as well as states of reduced or absent sensitivity to noxious stimuli. The term analgesia encompasses the term “antinociception”, which is used in the art as a quantitative measure of analgesia or reduced pain sensitivity in animal models. In the context of the present disclosure, such states of reduced or absent pain perception are induced by the administration of an aster extract; optionally in combination with at least one opioid drug. The term “enhancing the analgesic effect”, as used herein means that the duration and/or extent of the analgesic effect are improved over a suitable control.

The term “opioid” generally refers to a compound that binds to opioid receptors. As used herein, the term “opioid drug” encompasses all natural and synthetic opioids. For example, opioid drugs to compounds with morphine-like, painkilling (analgesic) effect. However, the present invention is not limited thereto; rather, opioid drugs may also include drugs acting on opioid receptors present in the central nervous system and/or peripheral system, as well as those acting on opioid receptors present in the gastrointestinal tract. Illustrative examples of natural opioid include, but are not limited to, morphine, code)ne, thebaine, and salvinorin A. Synthetic opioids include semi-synthetic opium alkaloids derivatives such as heroin (diacetylmorphine), dihydrocodeine, hydromorphone, nicomorphine, and oxycodone. Illustrative examples of fully synthetic opioid drugs include, but are not limited to, anilidopiperidines (e.g., fentanyl), phenylpiperidines (e.g., pethidine), diphenylpropylam)ne derivatives (e.g., loperamide), benzomorphan derivatives (e.g., dezocine), oripavine derivatives e.g., buprenorphine), and morphinan derivatives (e.g., butorphanol).

As used herein, the term “fresh” refers to plant components that have not (yet) been processed, or only minimally processed (e.g., cut or sliced and/or packaged) after harvest and which are not preserved by substantive drying. Furthermore, the term “fresh” does not necessarily require a strict time-dependency. Rather, it is used solely to differentiate between dried plant components and non-dried plant components.

As used herein the term “dried” refers to a range of moisture contents typically observed when a plant component is dehydrated. The drying can occur by any means known in the art, including sun drying, oven drying and freeze drying. Moisture contents in dried plant components can range from 1 to 20% by weight, however, typical ranges are between 2 and 5%.

The terms “extract from a Tatarian aster plant,” “plant extract,” or “aster extract” as used herein, refer to a composition prepared by contacting plant components from the Tatarian aster plant with a solvent following standard procedures such as those described herein. As could be appreciated, the term “plant extract” encompasses crude extracts as well as processed or refined extract. Specifically, crude extracts are prepared by a simple extraction in which selected plant components are contacted with at least one extractant. In some optional cases, the thus-obtained crude extracts are subjected to one or more separation and/or purification steps to obtain processed or refined extracts. The plant extract may be in liquid form, such as a solution, concentrate, or distillate; or it may be in solid form in which the solvent is removed, such as in granulate or powder form.

The prior research conducted by the present inventors reveals that the aster extract is effective in treating opioid-induced constipation (see, U.S. patent publication 2012/0164251, the entirety of which is incorporated herein by reference). Opiod-induced constipation (OIC) is the most common side effect experienced by patients receiving opioid therapy. Accordingly, additional medicines are often required to be prescribed along with the opioid painkillers that cause the OIC. For example, oral opioid-receptor antagonists, including naloxone, naltrexone, and nalmefene have been suggested to be potentially helpful in ameliorating opioid effects in the gastrointestinal tract. However, these agents also cross the blood-brain barrier and hence reverse analgesic effects of opioids. Methylnaltrexone is a newer agent that blocks peripheral opioid receptors. Therefore, rnethylnaltrexone may decrease the constipating effects of opioid pain medications in the gastrointestinal tract without affecting analgesia effects in the central nervous system. However, since up to 60 percent of opioid analgesia may be mediated by opioid receptors on peripheral sensory neurons, methylnaltrexone would increase pain under such circumstances. Hence, most prior medicaments for treating OIC compromise the effect of opioid drugs on the central receptors that mediate analgesia. Based on this understanding, one of ordinary skill in the art would expect that the aster extract suitable for treating OIC would also adversely affect the analgesic effect of opioid drugs. Nonetheless, follow-up research by the present inventors suggests that the aster extract would not jeopardize the analgesic effect of opioids drugs. Further, we unexpectedly find that (1) the aster extract, when administered alone, results in analgesia; and (2) the combination administration of the aster extract and an opioid drug (e.g, morphine) would provide an enhanced analgesic effect, compared with the sole administration of the opioid drug.

In view of the foregoing, one aspect of the present invention pertains to a method for treating pain using the present aster extract. Alternatively, the present invention is related to the use of the present aster extract for the preparation of a medicament for the treatment of pain.

According to embodiments of the present invention, the method comprises administering to the subject an effective amount of an extract from a Tatarian aster (Aster tartoricus) plant.

In certain embodiments, the aster extract is given to the subject via oral administration. However, the present disclosure is not limited thereto.

According to embodiments of the present disclosure, the aster extracts suitable for use in treating pain are prepared in accordance with processes set forth hereinbelow. First, in an extraction step, plant components collected from Tatarian aster plants are extracted with an extractant to obtain an extraction mixture. Then, in a separation step, the extraction mixture is subjected to column chromatography or precipitation.

According to embodiments of the present invention, the plant component suitable for use in the extraction is a fresh or dried material collected from the rhizome and/or root of a Tatarian aster plant.

In some embodiments, the extractant is water; whereas in some other embodiments, the extractant is an alcoholic solution consisting of water and 10-95% (v/v) ethanol. For example, the alcohol solution may be any of 10, 15, 20, 25, 30, 35, 40, 45, 50, 55, 60, 65, 70, 75, 80, 85, 90, and 95% (v/v) ethanol.

In optional embodiments, the extraction mixture is filtered, concentrated, and/or dried before the separation step.

In one embodiment, the extraction mixture is subjected to a reduced-pressure concentration to obtain a concentrate, and then the concentrate is washed with water to obtain a precipitate, which is suitable for use in the preparation of the present medicament. Optionally, the precipitate may be dried to yield extract powders, which is also suitable for use in the preparation of the present medicament.

In some alternative embodiments, the extraction mixture is subjected to a column chromatography in which the column is eluted with water followed by at least one eluents to obtain at least one eluate. Optionally, the eluate is concentrated to reduce the volume of the eluate and remove the eluent from the concentrated eluate. Still optionally, the concentrated eluate is dried to yield extract powders. According to principles and spirits of the present invention, the eluate, concentrated eluate, and extract powders are respectively suitable for use in the preparation of the present medicament.

Non-limiting examples of the eluent for use in the column chromatography include, but are not limited to, 40-95% (v/v) ethanol, acetone, and 40-95% (v/v) ethanol with 0.1-1% (v/v) formic acid.

Another aspect of the present invention pertains to a method for enhancing the analgesic effect of an opioid drug in a subject in need thereof. Alternatively, the present invention is related to the use of the aster extract for the preparation of a medicament (or an adjuvant) capable of enhancing the analgesic effect of an opioid drug.

According to embodiments of the present disclosure, the method comprises administering to the subject (1) an analgesically effective amount of the opioid drug, and (2) an effective amount of the aster extract for enhancing the analgesic effect of the opioid drug in the subject. Specifically, the aster extract for use in the present method is prepared in accordance with the preparation process provided in various embodiments of the present invention.

In certain embodiments, the aster extract is given to the subject via oral administration. However, the present disclosure is not limited thereto.

In optional embodiments of the present disclosure, the aster extract is administered prior to the administration of opioid drug.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Example 1

Preparation of Extracts from Tatarian Aster

### 1.1 Water Extraction

Dried, sliced components (roots and rhizomes) of Tatarian aster (purchased from Jin Han Hon Co., Ltd., Kaohsiung, Taiwan) were boiled and refluxed in reverse osmotic (RO) water in a ratio of 1:10 of Tatarian aster to water (w/w) for about 1 hour to obtain a crude extract. The crude extract was sifted through a 350-mesh sieve, and then filtered through a filter membrane having a pore size of 5 μm to give an aqueous water-extraction mixture. The water-extraction mixture was concentrated by freeze-drying to yield water-extracted powders.

### 1.1.1 Column Chromatography A

The water-extracted powders from example 1.1 were reconstituted in RO water and loaded onto a Diaion HP2O column (Diaion, Mitsubishi Chemistry Inc.) in which the weight ratio of the water-extracted powders to the dry column is about 1:30. Thereafter, the column was sequentially eluted by 3 bed volumes of RO water and 3 bed volumes of 95% (v/v) ethanol, and the eluate obtained from the column eluted with 95% (v/v) ethanol was collected. The eluate was concentrated to a suitable volume, and the ethanol was substantially removed from the concentrated eluate, which was later freeze-dried to yield eluted powders (hereinafter, Powder A).

### 1.1.2 Column Chromatography B

The water-extracted powders from example 1.1 were reconstituted in RO water and loaded onto a Diaion HP20 column (Diaion, Mitsubishi Chemistry Inc.) in which the weight ratio of the water-extracted powders to the dry column is about 1:20. Thereafter, the column was sequentially eluted by 3 bed volumes of RO water, 3 bed volumes of 40% (v/v), and 3 bed volumes of 95% (v/v) ethanol, and the eluate obtained from the column eluted with 95% (v/v) ethanol was collected. The eluate was concentrated to a suitable volume, and the ethanol was substantially removed from the concentrated eluate, which was later freeze-dried to yield eluted powders (hereinafter, Powder B).

1.2 50% (v/v) Ethanol Extraction

The extraction process of this example was similar to the water extraction described in example 1.1 except that 50% (v/v) ethanol was used as the extractant to obtain a 50% ethanol-extraction mixture. The 50% ethanol-extraction mixture was concentrated and freeze-dried to yield 50% ethanol-extracted powders.

### 1.2.1 Column Chromatography C

The 50% ethanol-extracted powders from example 1.2 were reconstituted in 50% ethanol and loaded onto a Diaion HP2O column (Diaion, Mitsubishi Chemistry Inc.) in which the weight ratio of the 50% ethanol-extracted powders to the dry column is about 1:20. Therea(ter, the column was sequentially eluted by 3 bed volumes of RO water, 3 bed volume of 95% (v/v) ethanol, 2 bed volumes of acetone, 2 bed volumes of 95% (v/v) ethanol containing 0.1% (v/v) formic acid, and 1 bed volume of 50% (v/v) ethanol containing 0.1% (v/v) formic acid to obtain respective eluates. The respective eluates obtained from the columns eluted with acetone, 95% (v/v) ethanol containing 0.1% (v/v) formic acid, and 50% (v/v) ethanol containing 0.1% (v/v) formic acid were collected, and combined. The combined eluate was then concentrated to a suitable volume, and the ethanol and acetone were substantially removed by evaporation, and the concentrate was subsequently freeze-dried to yield eluted powders (hereinafter, Powder C).

### Example 2

### Competitive Binding Assay

Of the three major classes of opioid receptors, mu (μ), delta (δ), and kappa (κ), the mu receptor has been proven to be the major target of opiate analgesics. In this experiment, competitive binding using the radiolabeled antagonist [³H]diprenorphine (³H-DPN) to investigate whether the present aster extract may specifically bind to the mu opioid receptor (MOR).

### 2.1 Materials and Methods

Membranes prepared from Chinese hamster ovary-K1 (CHO-K1) cells expressing recombinant human MOR were purchased from PerkinElmer Life and Analytical Sciences (Product No.: ES-542-M; K_{d}: 0.41 nM; Bₘₐₓ: 3.8 pmol per milligram of protein). ³H-DPN was also purchased from PerkinElmer Life and Analytical Sciences. Naloxone and TrisHCl were purchased from Sigma. The frozen membrane was aliquoted and stored at −80° C. for further use.

The membrane protein, 0.6 nM ³H-DPN, and 100 μg/mL or 30 μg/mL aster extract were incubated in reaction buffer (50 mM Tris-HCl, pH 7.4) at 25° C. for 60 minutes. The reaction mixture was then subjected to rapid vacuum filtration through GF/B filters pretreated with polyethyleneimine (0.3%) to quench the reaction, and then washed four times with 1 mL ice-cold Tris-HCl. The radiation intensity was detected by gamma-ray spectroscopy. Nonspecific binding was determined by the inclusion of reagents listed above plus a final buffer concentration of 10 μM naloxone hydrochloride. The non-specific binding data was subtracted to obtain specific binding data. The binding potency of the aster extract was expressed as the inhibition rate of the aster extract which was the percentage of the ³H-DPN that was displaced by the aster extract during the binding assay. Results are summarized in Table 1.

TABLE 1

| | | Concentration | Inhibition |
|---|---|---|---|
| Aster Extract | Batch No. | (μg/mL) | rate (%) |
| Powder A | PDC-1640 | 100 | 23 |
| Powder B | PDC-1764 | 100 | 83 |
| Powder C | PDC-2168 | 100 | 95 |
| Powder C | PDC-2168 | 30 | 63 |

As could be seen in Table 1, the present aster extracts bind to human mu opioid receptors. In particular, both aster extracts from working examples 1.1.2 (powder B) and L2.1 (powder C) exhibit high binding activity toward human mu opioid receptors.

### Example 3

### Tail Flick Test

Tail flick test was used to investigate the analgesic efficacy of the present aster extracts. Tail flick test measures the thermal nociceptive threshold defined as the latency required to elicit a tail response to heat. The typical non-treatment reaction time for a mouse subjected to this test was about 2-4 seconds. In treated animals, the latency to remove the tail lengthens in proportion to the is analgesic potency of the medicament.

### 3.1 Materials and Methods

Tatarian aster extracts were prepared in accordance with the method set forth in Example 1. Powders A and B were dispersed in water, respectively; whereas powder C was dispersed in carboxymethylcellulose (purchased from Sigma, USA). The compositions containing aster extract were administered at a dosage of 10 mL/kg, and the concentration of the active ingredient in each composition was adjusted according to the specified dosage in each test.

The experimental procedures were approved by the review board of Laboratory Animal Center, College of Medicine, National Taiwan University and conducted according to national animal welfare regulations.

Male ICR mice were purchased from National Taiwan University College of Medicine Laboratory Animal Center (Taiwan) and kept in an air-conditioned animal shelter at room temperature of 22° C. to 24° C. with controlled level of humidity (40% to 50%) in a 12-hour light-dark cycle. Each mouse weighed between 20 g to 25 g at the beginning of the test. Tap water and standard laboratory rodent chow provided ad libitum.

Tail flick test was performed using a Tail Flick Analgesia Meter (Columbus Instruments). Mice were given orally the aster extract (10 or 100 mg/kg) or vehicle (saline or 2% CMC) respectively on Day 1 (in the morning and afternoon) and Day 2 (in the morning), 30 minutes after the administration on Day 2, morphine was given by intraperitoneal injection (15 mg/kg) or subcutaneous injection (5 mg/kg). 20 minutes after the morphine injection, each mouse was placed on a platform and the tail was exposed to a focused beam of radiant heat. The heating intensity was adjusted to 15, and a 10-second cut-off was used to prevent burn or tissue damage.

Each mouse was subjected to the test three times.

Data generated during the tail flick test were then expressed as the percent maximal possible effect (MPE%), which is calculated according to equation (1):

MPE%=(test latency−pretest latency)/(cut-off (10 sec)−pretest latency)*100% (Equation (1)

All results are expressed as means ±SE; n refers to animals in each group. Differences between animals of each group were compared by one-way ANOVA followed by Dunnett's t test. A p value less than or equal to 0.05 was considered to be statistically significant.

### 3.2 Aster Extracts Enhance Analgesic Effect of Opioid Drugs

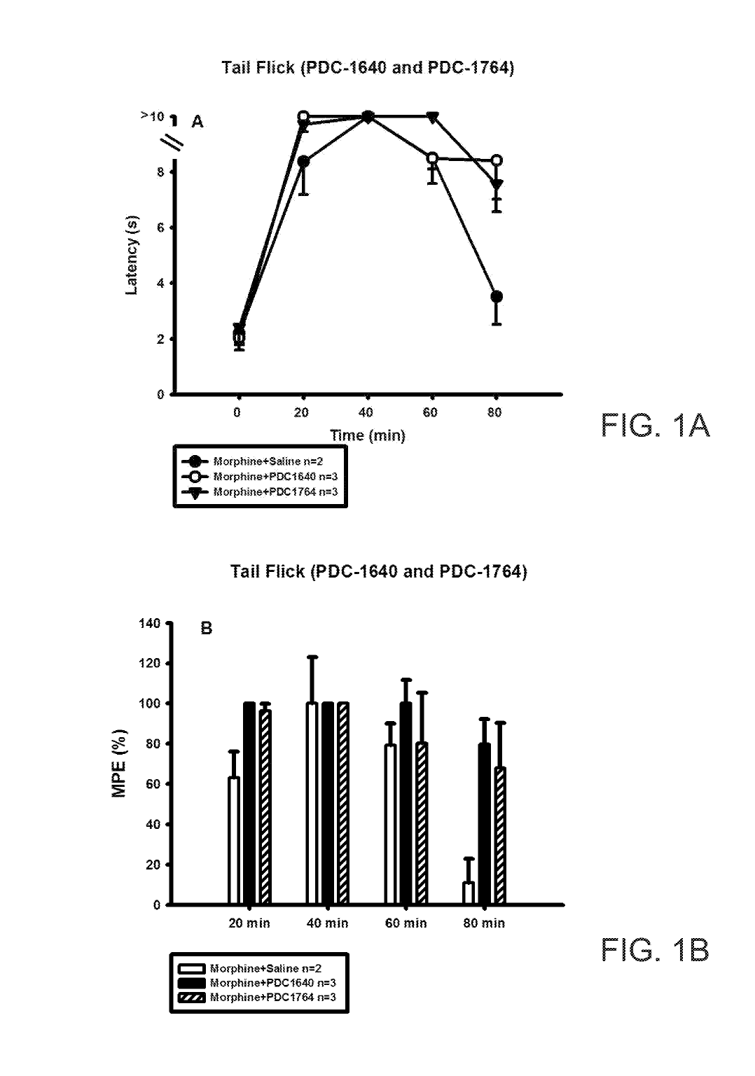

The line graph in FIG. 1A and bar graph in FIG. 1B respectively illustrate the time latency and MPE% determined from mice treated with (1) 100 mg/kg PDC-1640 (treatment group 1), (2) 10 mg/kg PDC-1764 (treatment group 2), or (3) saline (vehicle group 1), followed by intraperitoneal injection of 15 mg/kg morphine.

In this case, the analgesic effect induced by morphine took place in a relatively short term, and the present aster extracts did not adversely affect the analgesic effect of morphine. Moreover, at 60 minutes after morphine injection, significant increase in the latency time was observed, as compared to the treatment group 2 and the vehicle group 1 (FIG. 1A). Also, at 80 minutes after morphine injection, the latency times in both treatment group 1 and treatment group 2 is significantly higher than that of the vehicle group 1 (FIG. 1A).

Further, the analgesic effects induced by the combine administration of the present aster extracts and morphine were significantly higher than those obtained in their corresponding vehicle group 1 (FIG. 1B). Specifically, at 20 minutes after morphine injection, the MPE% of the vehicle group 1 was about 60%, whereas the respective MPE% in treatment groups 1 and 2 were 100%. Also, at 80 minutes after morphine injection, the vehicle group 1 exhibited a minimal analgesic effect (MPE% <20%), whereas the treatment groups 1 and 2 had considerable analgesic effects (MPE% >80% and >70%, respectively).

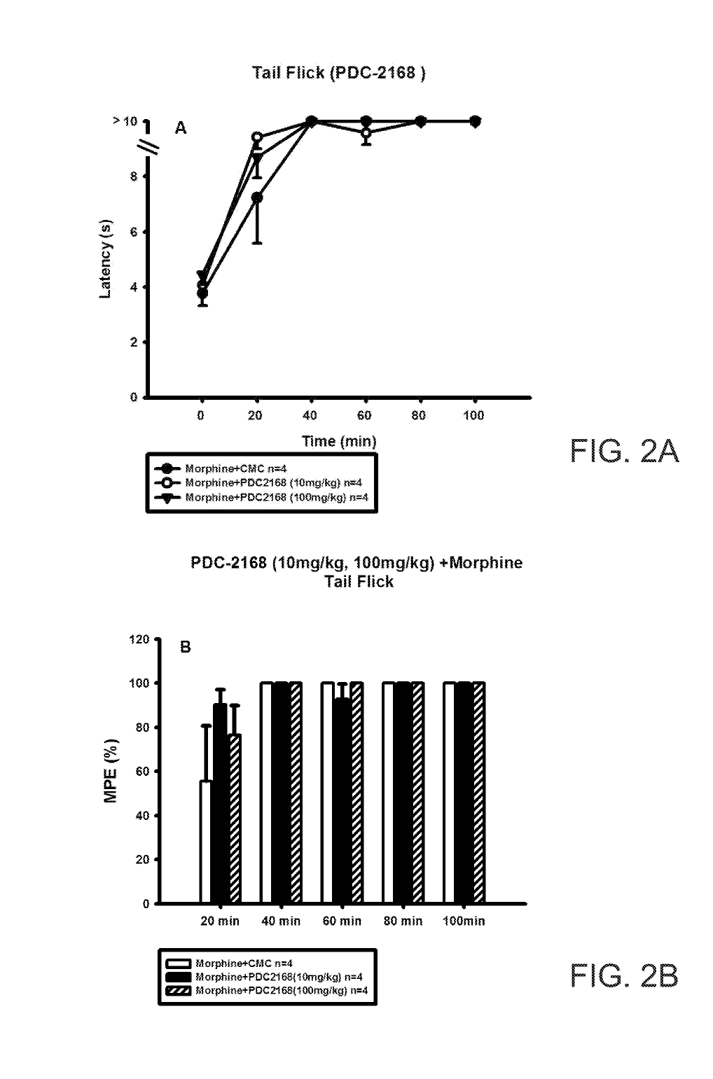

The aster extract of powder C (PDC-2168) was also tested for its potency in enhancing analgesic effect induced by morphine. The line graph in FIG. 2A and bar graph in FIG. 2B respectively illustrate the time latency and MPE% determined from mice treated with (1) 10 mg/kg PDC-2168 (treatment group 3), (2) 100 mg/kg PDC-2168 (treatment group 4), or (3) CMC (vehicle group 2), followed by intraperitoneal injection of 15 mg/kg morphine. As is evident from FIGS. 2A and 2B, merely marginal differences with respect to time latency was observed between the vehicle group 2 in comparison to treatment groups 3 and 4. Regarding the maximal possible effect, the administration of aster extracts moderately increased the MPE% from about 55% (vehicle group 2) to about 90% (treatment group 3) and 75% (treatment group 4), respectively, at 20 minutes after intraperitoneal injection of morphine.

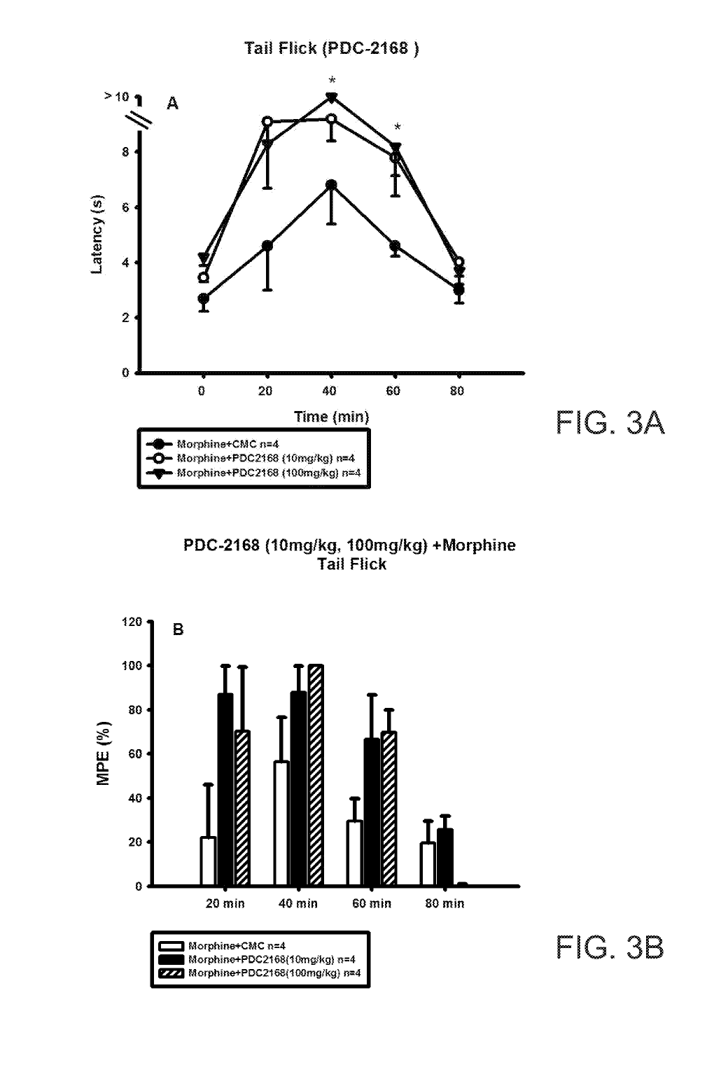

To further ascertain the effect of powder C (PDC-2168) on the analgesic effect of morphine, morphine was then administered subcutaneously to elicit a slower action of morphine. The line graph in FIG. 3A and bar graph in FIG. 3B respectively illustrate the time latency and MPE% determined from mice treated with (1) 10 mg/kg PDC-2168 (treatment group 5), (2) 100 mg/kg PDC-2168 (treatment group 6), or (3) CMC (vehicle group 3), followed by subcutaneous injection of 5 mg/kg morphine. In FIGS. 3A and 3B, significant increase in time latency was observed by comparing treatments groups 5 and 6 with the vehicle group 3; in particular at 40 and 60 minutes after morphine administration. With respect to the maximal possible effect, the co-administration of PDC-2168 substantially improved the MPE% in treatment groups 5 and 6, compared with the vehicle group 3.

In view of the foregoing, the present aster extracts (at least extracts of batch Nos. PDC-1640, PDC-1764, and PCD-2168) are capable of enhancing the analgesic effect induced by morphine by improving the short-term maximal possible effect in a subject. Also, these aster extracts prolong the duration of the analgesic effect induced by intraperitoneally or subcutaneously injected morphine, suggesting that the aster extracts are effective in enhancing the analgesic effect induced by morphine.

### 3.3 Aster Extracts Alone Possess Analgesic Effect

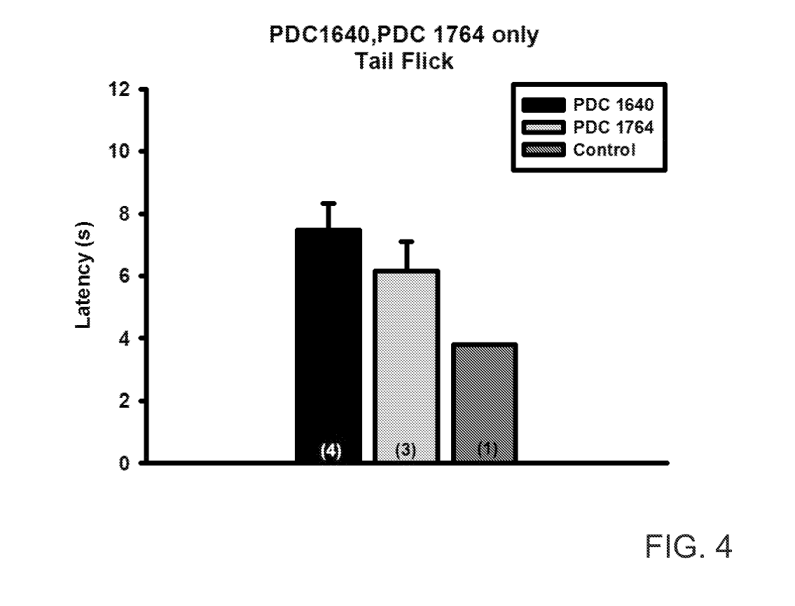

Here, the present aster extract was given to the mice without the co-administration of morphine to ascertain the analgesic effect of the present aster extract itself. FIG. 4 is a bar graph illustrating the time latency determined from mice treated with (1) 100 mg/kg PDC-1640 (treatment group 7), (2) 10 mg/kg PDC-1764 (treatment group 8), or (3) saline (vehicle group 4). The results summarized in FIG. 4 reveal that the present aster extracts (at least extracts of batch Nos. PDC-1640 and PDC-1764) are effective in delaying the tail flick in mice, suggesting that the aster extracts per se may act as an analgesic in mice.

These results indicate that Tatarian aster extracts prepared by the extractants and eluents described hereinabove are useful for treating pain for it may elicit analgesia by itself, as well as prolong the analgesic effect induced by opioid drugs such as morphine.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. Use of a solvent extract from a Tatarian aster (Aster tartaricus) plant for the preparation of a medicament for treating pain and/or enhancing the analgesic effect of an opioid drug in a subject in need thereof, wherein the solvent extract is prepared by a preparation method comprising extracting a plant component of Tatarian aster with an extractant to obtain an extraction mixture, and the plant component is selected from the group consisting of fresh rhizomes, fresh roots, dried rhizomes, and dried roots of the Tatarian aster.

2. The use of claim 1, wherein the extractant is water.

3. The use of claim 1, wherein the extractant is 10-95% (v/v) ethanol.

4. The use of claim 1, wherein the preparation method further comprises subjecting the extraction mixture to a column chromatography by eluting the column in sequence with water and at least one eluent selected from the group consisting of 40-95% (v/v) ethanol, 40-95% (v/v) ethanol with 0.1-1% (v/v) formic acid, and acetone.

5. The use of claim 4, wherein the extraction mixture is filtered, concentrated, or dried before being subjected to the column chromatography.

6. The use of claim 4, wherein the preparation method further comprises concentrating the eluate of column chromatography to reduce the volume of the eluate and remove the eluent therefrom.

7. The use of claim 1, wherein the preparation method comprises, extracting the dried roots and/or rhizomes of the Tatarian aster with water to produce an extraction mixture; and
subjecting the extraction mixture to a column chromatography by eluting the column in sequence with water and 95% (v/v) ethanol, and collecting the eluate obtained from the column eluted by the 95% (v/v) ethanol.

8. The use of claim 1, wherein the preparation method comprises,
extracting the dried roots and/or rhizomes of the Tatarian aster plant with water to produce an extraction mixture; and
subjecting the extraction mixture to a column chromatography by sequentially eluting the column with water, 40% (v/v) ethanol, and 95% (v/v) ethanol, and collecting the eluate obtained from the column eluted by the 95% (v/v) ethanol.

9. The use of claim 1, wherein the preparation method comprises, extracting the dried roots and/or rhizomes of the Tatarian aster plant with 50% (v/v) ethanol to produce an extraction mixture; and
subjecting the extraction mixture to a column chromatography by sequentially eluting the column with water, 95% (v/v) ethanol, acetone, 95% (v/v) ethanol containing 0.1% (v/v) formic acid, and 50% (v/v) ethanol containing 0.1% (v/v) formic acid; and collecting and combining the eluates respectively obtained from the column eluted by the acetone, the 95% (v/v) ethanol containing 0.1% (v/v) formic acid, and the 50% (v/v) ethanol containing 0.1% (v/v) formic acid.

10. A method for treating pain in a subject in need thereof, comprising administering to the subject an effective amount of a solvent extract of a Tatarian aster plant to treat pain, wherein the solvent extract is prepared by a preparation method comprising extracting a plant component of Tatarian aster with an extractant to obtain an extraction mixture.

11. The method of claim 10, wherein the extractant is water.

12. The method of claim 10, wherein the extractant is 10-95% (v/v) ethanol.

13. The method of claim 10, wherein the preparation method further comprises subjecting the extraction mixture to a column chromatography by eluting the column in sequence with water and at least one eluent to obtain at least one eluate, wherein the at least one eluent is selected from the group consisting of 40-95% (v/v) ethanol, 40-95% (v/v) ethanol with 0.1-1% (v/v) formic acid, and acetone.

14. The method of claim 13, wherein the extraction mixture is processed by at least one of filtration, concentration, and drying before being subjected to the column chromatography.

15. The method of claim 13, wherein the preparation method further comprises concentrating the eluate to reduce the volume of the eluate and remove the eluent therefrom.

16. The method of claim 10, wherein the preparation method comprises, extracting the dried roots and/or rhizomes of the Tatarian aster with water to produce an extraction mixture; and
subjecting the extraction mixture to a column chromatography by eluting the column in sequence with water and 95% (v/v) ethanol, and collecting the eluate obtained from the column eluted by the 95% (v/v) ethanol.

17. The method of claim 10, wherein the preparation method comprises, extracting the dried roots and/or rhizomes of the Tatarian aster plant with water to produce an extraction mixture; and subjecting the extraction mixture to a column chromatography by sequentially eluting the column with water, 40% (v/v) ethanol, and 95% (v/v) ethanol, and collecting the eluate obtained from the column eluted by the 95% (v/v) ethanol.

18. A method for enhancing the analgesic effect of an opioid drug in a subject in need thereof, comprising administering to the subject an analgesically effective amount of the opioid drug and an effective amount of a solvent extract of a Tatarian aster plant to enhance the analgesic effect of the opioid drug in the subject, wherein the solvent extract is prepared by a preparation method comprising extracting a plant component of Tatarian aster with an extractant to obtain an extraction mixture.

19. The method of claim 18, wherein the solvent extract of the Tatarian aster plant is administered prior to the administration of the opioid drug.

20. The method of claim 18, wherein the opioid drug is morphine.

21. The method of claim 18, wherein the extractant is water.

22. The method of claim 18, wherein the extractant is 10-95% (v/v) ethanol.

23. The method of claim 18, wherein the preparation method further comprises subjecting the extraction mixture to a column chromatography by eluting the column in sequence with water and at least one eluent to obtain at least one eluate, wherein the at least one eluent is selected from the group consisting of 40-95% (v/v) ethanol, 40-95% (v/v) ethanol with 0.1-1% (v/v) formic acid, and acetone.

24. The method of claim 23, wherein the extraction mixture is processed by at least one of filtration, concentration, and drying before being subjected to the column chromatography.

25. The method of claim 23, wherein the preparation method further comprises concentrating the eluate to reduce the volume of the eluate and remove the eluent therefrom.

26. The method of claim 18, wherein the preparation method comprises,
extracting the dried roots and/or rhizomes of the Tatarian aster plant with 50% (v/v) ethanol to produce an extraction mixture; and
subjecting the extraction mixture to a column chromatography by sequentially eluting the column with water, 95% (v/v) ethanol, acetone, 95% (v/v) ethanol containing 0.1% (v/v) formic acid, and 50% (v/v) ethanol containing 0.1% (v/v) formic acid, and collecting and combining the eluates respectively obtained from the column eluted by the acetone, the 95% (v/v) ethanol containing 0.1% (v/v) formic acid, and the 50% (v/v) ethanol containing 0.1% (v/v) formic acid.
